(19) United States
(12) Patent Application Publication
(10) Pub. No.: US 2026/0242853 A1
(43) Pub. Date: Aug. 20, 2026

(54) IMPROVED METHOD FOR DETECTING A TARGET VARIANT BASE USING A VARIANT-SPECIFIC PROBE

(71) Applicant: RARITY BIOSCIENCE AB, Uppsala (SE)

(72) Inventor: Lei CHEN, Storvreta (SE)

(73) Assignee: RARITY BIOSCIENCE AB, Uppsala (SE)

(21) Appl. No.: 19/162,652

(22) PCT Filed: Mar. 8, 2024
(86) PCT No.: PCT/EP2024/056203

§ 371 Date: Sep. 5, 2025

(30) Foreign Application Priority Data
Mar. 8, 2023 (GB) 2303359.0

(51) Int. Cl. C12Q 1/6827; C12Q 1/34; C12Q 1/6841; C12Q 1/6855; C12Q 1/6858; C12Q 1/6876

(52) U.S. Cl. CPC C12Q 1/6827; C12Q 1/34; C12Q 1/6841; C12Q 1/6855; C12Q 1/6858; C12Q 1/6876; C12Y 301/00; C12Q 2600/156

(57) ABSTRACT

The present invention provides a method of detecting a target variant nucleotide in a target nucleotide sequence in a sample, said method comprising contacting said target nucleotide sequence with a variant-specific padlock probe capable of hybridising to said target nucleotide sequence, and detecting said probe in order to detect said target variant nucleotide wherein the variant-specific padlock probe comprises a variant-specific nucleotide at a 3′ end thereof, and wherein said variant-specific 3′ nucleotide is a modified nucleotide.

### FIELD

The present disclosure and invention relate to improvements in the use of allele-specific, or more generally variant-specific, probes to detect a specific allele, or variant base. In particular, provided herein are modified variant-specific probes, and their use in detecting a target variant nucleotide in a target nucleic acid sequence. In accordance with the present methods and uses, the discrimination of different variants of a nucleotide at a target position in the target sequence, or more generally of different target sequence variants, may be improved.

### BACKGROUND

The detection of target nucleic acid sequences has applications in many different fields, including notably clinically, for personalised medicine and in the diagnosis, prognosis and/or treatment of disease, such as cancer, infectious diseases and inherited or genetic disorders, as well as in research and biosecurity.

In particular, it is frequently desired to be able to detect individual variants of a target sequence, such as particular alleles, or mutations, and thus to discriminate, or distinguish between such variants. Target nucleic acid sequences may commonly occur in variant forms, for example, allelic variants or polymorphisms, or mutant and wild-type sequences, and it may be desirable to detect which variant is present, notably in the case of mutation detection, including in the case of disease. To this end, so-called “allele-specific” forms of nucleic acid detection have been developed—in this case, the term “allele-specific” is used generically and broadly to refer to all forms of variants and mutations, and not just strictly “alleles” in the inherited sense (i.e. it refers to any variant, and so may alternatively be termed “variant-specific”). Such methods generally use “allele-specific” probes, and more particularly allele-specific hybridisation probes, which are designed to hybridise specifically to one particular variant.

In particular, although their use is not limited to such variants, such hybridisation probes may be used to discriminate between target sequences which differ in only a single base, i.e. they can be used to detect a single variant base in a target nucleotide sequence. Thus, a variant-specific probe may comprise a nucleotide which is specific for, or designed to hybridise specifically to, a target nucleotide in a target sequence, and which determines whether or not a probe will hybridise to a test sequence (i.e. hybridisation will only occur if the target nucleotide is present). Typically, such a “variant-specific” nucleotide is located at the 3′ end of the probe.

Allele-specific assays are widely known and described in the art, and can take various forms, using various probe formats, e.g. simple hybridisation probes (e.g. FISH probes), ligation-based probes, including e.g. oligonucleotide ligation assays (OLA) using two or more ligation oligonucleotides or padlock probes, or probes which are subject to extension/polymerisation reactions following hybridisation.

In all cases, there can be circumstances where hybridisation of the probes is not as specific as desired, and a clear distinction between alleles, or variants, is not achieved. In particular, different base pairs may have different hybridisation strengths or affinities, and this can affect assay specificity. For example, a G to C linkage is stronger than an A to T linkage, and a probe comprising 3′ terminal A as the variant-specific nucleotide has weaker affinity for its target (comprising T as the variant base) compared to a probe comprising a 3′ terminal G as the 3′ terminal variant-specific nucleotide for its target (comprising C as the variant base). Thus, there is an observable tendency for G-probes (i.e. C-specific probes) to hybridise, or contribute to signal, more than A-probes (i.e. T-specific probes). Indeed, it has been observed that a G-probe may in some situations hybridise incorrectly to a T-containing target (where T, not C, is the variant nucleotide), leading to non-specific background signal.

There is thus a need for improved methods for allele-specific, or in other words, variant-specific, detection of nucleic acids. This is addressed by the methods, uses and probes herein.

### SUMMARY

The present inventors have determined that by incorporating modified nucleotides, including modified inter-nucleotide linkages, into variant-specific hybridisation probes, particularly padlock probes, discrimination between variants in detection assays for nucleic acid targets comprising target variant nucleotides may be improved.

Thus, for example, the specificity of variant-specific probes in a detection method where different probes are used to detect the presence of different variants in a target sequence may be increased. Thus, a better distinction between different variants may be obtained.

The modification may be introduced to alter the binding affinity of the nucleotide in question to its target binding partner (i.e., its target or complementary nucleotide) in the target sequence. Binding affinity may be increased or decreased to even out differences in affinity between different probes. In this manner, variant distinction may be improved.

In particular, the variant-specific nucleotide which lies at the 3′ terminal of the variant-specific hybridisation probe is modified.

Accordingly, in a first aspect, provided herein is a method of detecting a target variant nucleotide in a target nucleotide sequence in a sample, said method comprising contacting said target nucleotide sequence with a variant-specific probe capable of hybridising to said target nucleotide sequence, and detecting said probe in order to detect said target variant nucleotide, wherein the variant-specific probe comprises a variant-specific nucleotide at its 3′ end, and wherein said variant-specific 3′ nucleotide is a modified nucleotide.

In this aspect, and in the aspects below, and as noted further below, the variant-specific probe is particularly a padlock probe.

In a second aspect, provided herein is use of a variant-specific probe to detect a target variant nucleotide in a target nucleotide sequence, wherein the variant-specific probe comprises at least one target-binding site that is complementary to the target nucleotide sequence and is capable of hybridising thereto, and wherein said target binding site lies at the 3′ end of the probe and comprises a variant-specific nucleotide at its 3′ end which is a modified nucleotide.

Also provided herein, in a third aspect, is a variant-specific probe comprising at least one target-binding site that is complementary to a target nucleotide sequence and is capable of hybridising thereto, wherein said target binding site lies at the 3′ end of the probe and comprises a variant-specific nucleotide at its 3′ end which is capable of base-pairing with a target variant nucleotide in the target nucleotide sequence, and which is a modified nucleotide.

A fourth aspect provides a kit for use in a method as defined above, said kit comprising a plurality of variant-specific probes, each specific for a different variant at a target position in a target-nucleotide sequence, wherein at least one variant-specific probe comprises a modified nucleotide at its 3′ end.

In particular, in all these aspects, the variant-specific probe may be defined as comprising a nucleotide at its 3′ end which
(i) is capable of discriminating the target variant nucleotide from a non-target nucleotide; and
(ii) is a modified nucleotide.

In other words, the 3′ end nucleotide of the variant-specific probe is capable of base-pairing with, or hybridising to, the target variant nucleotide. Put another way, the variant-specific probe comprises a nucleotide at the 3′ end that is complementary to the target nucleotide in the target nucleic acid sequence. That is, the 3′ end nucleotide (i.e. the 3′-most nucleotide) of the probe is complementary to the target nucleotide in the target nucleotide sequence (i.e. to the variant base).

Since the specificity of the probe is determined by at least the 3′ terminal nucleotide of the probe, this may be referred to as the “genotyping base” or “genotyping nucleotide” of the probe. Accordingly, the method may also be referred to as a method of genotyping a target nucleotide in a target nucleotide sequence, or more particularly as a method of genotyping a target nucleotide (or base) at a target position in a nucleic acid sequence.

As noted above, the modified nucleotide may comprise a modification which alters its binding affinity to the nucleotide in the target nucleic acid sequence at the position of the variant nucleotide. Binding affinity may be increased or decreased.

In an embodiment, the probe comprises one or more additional modified nucleotides (i.e. in addition to the 3′ end modified nucleotide). For example, the penultimate and/or the third to last nucleotide is additionally modified.

In another embodiment, the probe does not comprise any further modified nucleotides beyond the 3′ end modified nucleotide.

The modification may be any modification which alters the affinity of a nucleotide for its complementary base, and includes modifications to the sugar and/or base of the nucleotide and/or the backbone. By the latter is meant a modification to the linkage which joins the nucleotide to an adjacent nucleotide. Accordingly, in an embodiment, the modification can be a modified inter-nucleotide linkage. In an embodiment, the modification is a phosphorothioate or phosophorothiolate linkage.

The variant target nucleotide may be a single nucleotide variant or it may be part of a longer variant sequence, or may occur as the result of a deletion. It may be a naturally occurring variant, e.g. an allelic variant or polymorphism, or it may be a mutation. Thus, the variant base may be artificially introduced.

The target nucleotide sequence occurs, or is present in, a target nucleic acid molecule in the sample. In an embodiment, the sample is a clinical sample, for example, a liquid biopsy sample, e.g. a blood-derived sample.

In an embodiment, the target nucleic acid is a naturally occurring nucleic acid, e.g. genomic DNA, or an amplicon or copy thereof. In another embodiment, it is RNA or cDNA, or a copy or amplicon thereof.

In an embodiment, the sample is a cell or tissue sample and the target variant nucleotide is detected in situ.

The method may be performed in multiplex. In an embodiment, the method is used or performed to detect two or more variants of a nucleotide at a target position in a nucleotide sequence, or more particularly, which of two or more different target variant nucleotides are present at a target position in the target nucleotide sequence. In such a method, two or more different variant-specific probes are used, one for each target variant nucleotide to be detected, and wherein the 3′ end nucleotide of at least one of said variant-specific probes is modified.

Accordingly, in such an embodiment, the method may be seen as a method for discriminating, or distinguishing, different variants of a nucleotide at a target position in a target nucleotide sequence, said method comprising contacting said target nucleotide sequence with two or more different variant-specific probes, each capable of hybridising to said target nucleotide sequence and detecting a different variant at the target position, and detecting one or more of said probes in order to detect the target variant nucleotide which is present, wherein the variant-specific probes each comprise a different variant-specific nucleotide at their 3′ ends, and wherein said variant-specific 3′ nucleotide of at least one of the probes is a modified nucleotide.

The variant-specific probe may be a hybridisation probe or a ligation and/or extension probe. In an embodiment, the variant-specific probe is a circularisable probe, and more particularly it is a padlock probe. In such an embodiment where the probe is a circularisable probe (or a padlock probe), the method herein may further comprise a step of degrading any probes which have not been circularised, in particular using an enzyme with 5′ exonuclease activity.

### DESCRIPTION OF DRAWINGS

FIG. 1 shows the use of non-phosphorothioate bond modified probes (V4) vs Phosphorothioate bond modified probes (V42) detecting the same IDH1 p.R132C sample. Wild-type and mutant populations were detected in conditions of 59° C. by the unmodified V4 probes or the modified V42 probes. Wild-type and mutant populations are also detected in a condition of 61° C. by the unmodified V4 probes. The results are shown in FACS plots which are gated on the left (mutant population) and on the bottom right (wild-type population).

FIG. 2 shows the use of non-phosphorothioate bond modified probes (V4) vs Phosphorothioate bond modified probes (V42) detecting the same IDH1 p.R132H sample. Wild-type and mutant populations are detected in conditions of 59° C. by the unmodified V4 probes or the modified V42 probes. Wild-type and mutant populations are also detected in a condition of 61° C. by the unmodified V4 probes. The results are shown in FACS plots which are gated on the left (mutant population) and on the bottom right (wild-type population).

### DETAILED DESCRIPTION

The methods and uses herein enhance distinction between different variants in the detection of variant nucleotide sequences. More particularly, the methods and uses herein address the problems caused in such detection assays by differences in the hybridization, e.g. in hybridization strength, of different probes which are used to detect different variants of the target nucleotide sequence (i.e. differences in the hybridization to their respective targets of probes directed to different variants).

For example, in some cases it has been observed that probes designed to detect particular variant nucleotides may be more prone to exhibit greater non-specific binding. Thus, greater off-target hybridization may occur with some probes designed to detect particular variant nucleotides than with others of differing affinities. For example, a probe containing a 3′ terminal G (designed to detect C as a variant nucleotide) may exhibit off-target hybridization to a sequence containing T at the target position, and this may in some circumstances be stronger than that of the probe which is designed to detect a T variant nucleotide.

Whilst not wishing to be bound by theory, it is hypothesized that these differences in the hybridization of variant-specific probes specific for different target variant nucleotides may arise from differences in the respective affinities of different nucleotides for their respective complementary bases. However, this hypothesis does not preclude that other factors are at play in the improved distinction that is observed, and that that may be achieved, with the modified variant-specific probes herein.

As noted above, it is frequently desired to be able to detect individual variants of a target sequence, and thus to discriminate, or distinguish, between such variants.

At its broadest, a variant sequence is simply a sequence which comprises or contains one or more sequence differences compared to a reference or comparator sequence (i.e. at one or more positions in the sequence, there is a different nucleotide (or in other words, a different base is present at that position). A target nucleotide sequence is a sequence in which it is desired to detect a variant nucleotide. As noted above, target nucleic acid sequences may commonly occur in variant forms, for example, allelic variants or polymorphisms, or mutant and wild-type sequences and it may be desirable to detect which variant is present, for example, wild-type or mutant sequences, or which of a number of different possible mutations, or to detect a new mutation or variation. A variant nucleotide is a nucleotide at a given position in a target sequence (a “target position”) which may vary (be different) in different variants of the target sequence. The target variant nucleotide is the variant nucleotide it is desired to detect. The variant nucleotide may be a single nucleotide variation, or it may occur in the context of a longer variant sequence, i.e. there may be a stretch of nucleotides in the target sequence which contains one or more contiguous or non-contiguous variant nucleotides, and the target variant nucleotide may be any one of these. Thus, the variant sequence can be a longer sequence, or a portion or part of a target sequence which contains one or more variant nucleotides. For example, a variant sequence (or alternatively termed, a “sequence variant”) may be a modified sequence, in terms of having a different sequence of nucleotides, or it may comprise additions, insertions or deletions of one or more nucleotides. All types of mutation and sequence rearrangements are included. Thus, variant sequences can be of the same length or different lengths.

As noted above, the term “variant” is used synonymously with the term “allele” to include any variant, howsoever it arises or occurs, and analogously, the terms “variant-specific” and “allele-specific” are interchangeable herein.

Variant-specific probes designed to detect or discriminate between different variants commonly comprise a nucleotide which is specific for (i.e. complementary to) a target nucleotide in a target sequence, and which determines whether or not a probe will hybridise to a test sequence (i.e. hybridisation will only occur if the target nucleotide is present). Typically, and in the probes herein, such a “variant-specific” nucleotide is located at the 3′ end of the probe (i.e. it is the last nucleotide at the 3′ end of the probe or of a part thereof). This is referred to as the 3′ end nucleotide, and may also be referred to as the 3′ most nucleotide or 3′ terminal nucleotide.

Natural differences in the affinity of the nucleotides in the base-pairings A-T and C-G mean that probes designed to detected different variants (e.g. to detect T at the target position versus C, meaning a probe with 3′ terminal A compared to a probe with 3′ terminal G) can have different affinities for their respective targets (an “A” probe having lower affinity than a “G” probe). As discussed above, these differences can negatively impact assay performance, and in many cases may lead to loss of specificity. Non-specific binding or probes may occur, particularly of probes with a 3′ G (so-called “G-probes”).

In the present probes, uses and methods, it has been found that differences in the hybridisation ability, or hybridisation strength or hybridisation specificity, between different probes may be compensated for by modifying the 3′ end nucleotide of variant-specific probes.

In particular, whilst not wishing to be bound by theory, it is proposed that differences in affinity may be evened out, by modifying the 3′ end nucleotide of variant-specific probes to alter their affinity to their target.

It is not required that each variant-specific probe used in a method to detect different variants is modified, but at least one probe is modified. This will depend upon the variants being detected, and the probes which are used. For example, in detecting possible T and C variants of a target nucleotide, the G-probe used to detect the C-variant will have a higher hybridisation strength (e.g. affinity) than the A-probe used to detect the T-variant. To even out, or compensate for, this difference in hybridisation strength (e.g. affinity), the G-probe may be modified to reduce its hybridisation strength (e.g. affinity) for the C-variant-containing target sequence. The A-probe may be unmodified. Conversely, the A-probe may be modified to increase its affinity and the G-probe may be unmodified. In another embodiment, both probes may be modified. If more than 2 probes are being used, it may be advantageous to modify more than one probe.

Indeed, irrespective of the mechanism which is operating, by modifying the 3′ end nucleotide of the variant-specific probes, the ability of the probe to bind specifically to its target variant sequence, and to discriminate between different target variant nucleotides is improved. Thus, the specificity of a variant-specific probe may be improved.

Modifications which alter the hybridisation of nucleotides, or the affinity of a nucleotide for its complementary base, or which when the nucleotide is modified in the context of a nucleotide sequence, may alter the hybridisation strength of that sequence to a complementary sequence, are known in the art. Such modifications may increase or decrease (i.e. weaken) affinity or hybridisation strength. The modification may take various forms, as is well known in the art.

As used herein, the term “modified nucleotide” broadly refers to a nucleotide which is not found in nature in the context of nucleic acid molecules as they occur in living organisms, i.e. an unnatural or non-native nucleotide. It may be defined as not being a nucleotide as occurs in nature in nucleic acid/DNA. The modification may be in the structure of the nucleotide, including in the base and/or sugar, or it may be a so-called backbone modification, where the backbone of the nucleotide sequence is modified, or in other words the linkage between nucleotides is altered (in the case of the 3′ end nucleotide, this is the linkage between the 3′ most nucleotide and the 3′ penultimate nucleotide). Thus, the modified nucleotide may be a nucleotide linked to its adjacent nucleotide by an unnatural bond (that is a bond which is not a phosphodiester bond).

In terms of modified nucleotides with a modified structure, a wide variety of these are known in the art, including substituted nucleotides (that is nucleotides comprising one or more modifying chemical groups on the base and/or sugar moieties) and synthetic modified nucleotides, or nucleotides with unnatural or synthetic bases.

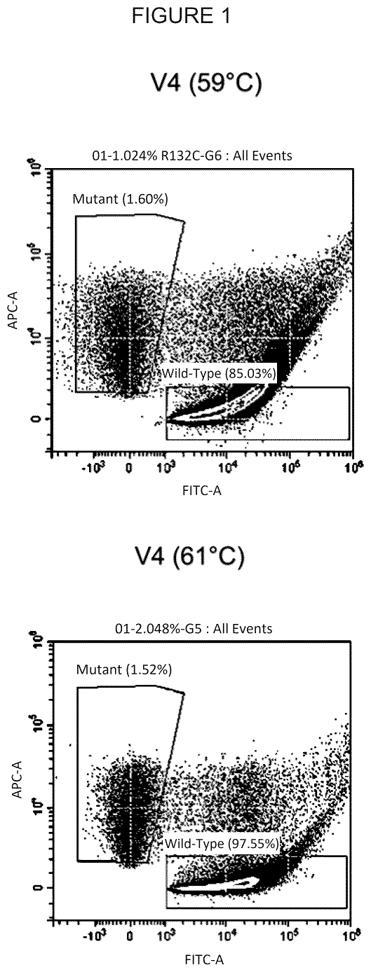

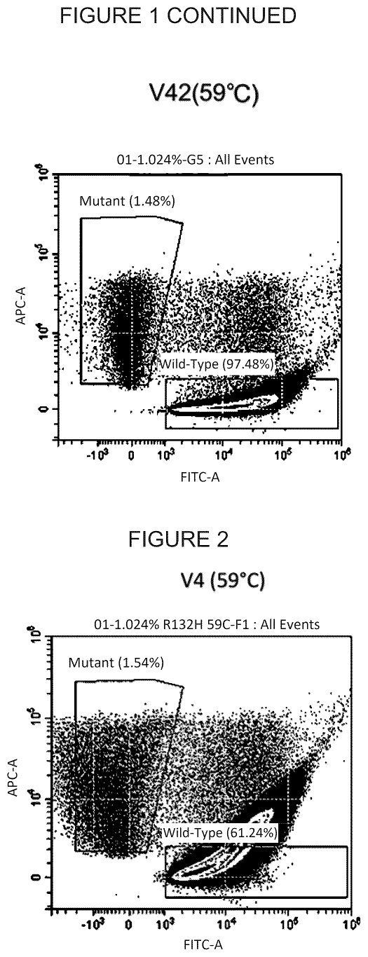

For example, a range of modified nucleotides are described in Duffy et al., BMC Biology 2020, 18, 112 and Ochoa and Milam, Molecules 2020, 25, 4659 (see e.g. FIG. 1 thereof), both of which are incorporated herein by reference. Any of the modified nucleotides described or mentioned in these documents, or elsewhere in the art, or listed below, may be used.

Nucleotides with sugar modifications reported in the art include: 2′F RNA; 2′OMe RNA; LNA; FANA; HNA; or 2′-O-methoxy-ethyl(2′ MOE) bases.

Nucleotides with modifications in the base include: C7-modified deaza-adenine; C7-modified deaza-guanosine; C5-modified cytosine; or C5-modified uridine; wherein C7 and/or C7 of the base may be substituted with H, CI or F or other groups; deoxyinosine; 5-hydroxybutynyl-2′-deoxyuridine (Super T®); 8-aza-7-deazaguanosine (Super G®); 2,6-diaminopurine (Z).

Still other modified nucleotides with modified bases include: dZ; dP; dS; dB; Ds; Px; Pa; 5SICSN; aM; TPT3; or NaM. However, in certain embodiments, these are less preferred.

Backbone modifications, or modified nucleotide linkages, include modifications to the phosphate group in the phosphodiester linkage, including phosphorothioate or phosphorothiolate linkages, or borano-phosphate linkages. In the phosphorothioate modification, a non-bridging oxygen covalently bound to phosphorous is replaced by a sulphur atom (as shown in structure A below). Analogously, in the borano-phosphate modification, a non-bridging oxygen is replaced with BH₃. In the phosphorothiolate modification (also known as a 5′-thio nucleoside), a sulphur atom replaces the 5′-bridged oxygen connected to the sugar moiety (as shown in structure B below).

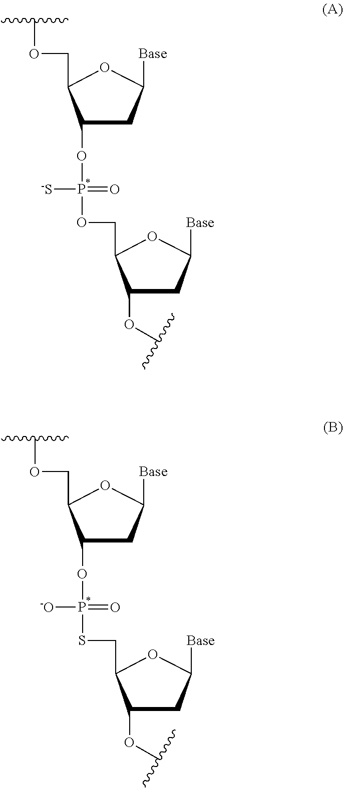

Other known backbone modifications which may be used include phNA (methyl phosphonate, where a non-bridging oxygen in the phosphate linkage is replaced by CHs) or peptide nucleic acid (PNA).

Other backbone (or linkage) modifications include those which involve the sugar moiety of the nucleotide (i.e. so-called sugar/backbone modifications). Such modifications known and described in the art include mirror DNA, ribuloNA, TNA, tPhoNA and dXNA.

Representative exemplary modified nucleotides which may be used include:
(a) phosphorothioate or phosphorothiolate;
(b) LNA;
(c) 2′-O-methoxy-ethyl(2′MOE);
(d) 2,6-diaminopurine (Z);
(e) deoxyinosine;
(f) 5-methyl dC;
(g) 5-hydroxybutynyl-2′-deoxyuridine (Super T®); or
(h) 8-aza-7-deazaguanosine (Super G®).

Phosphorothioate or phosphorothiolate modifications are preferred according to the methods, uses and modified probes etc. herein.

It is required that the 3′ end nucleotide (the discriminatory nucleotide) of at least one probe provided herein or used in the methods herein is modified. However, it is not precluded that other nucleotides in the probe are also modified. Thus, the probe may comprise one or more modified nucleotides, for example, 2 or 3 or more (these will include the 3′ end nucleotide). For example, the last 2 or 3 nucleotides of the probe may be modified, or any one or more of the 6 nucleotides from the 3′ end of the probe, including at least the 3′ end nucleotide, may be modified. As will be described in more detail below, a variant-specific probe as provided or used herein may be provided in one or more parts, and accordingly a reference herein to the probe includes a part thereof.

The target nucleotide sequence may be any target sequence it is desired to detect. In this regard, the term “nucleotide sequence” is used interchangeably with “nucleic acid sequence”.

The term “detecting” is used broadly herein to include any means of determining the presence of the target variant nucleotide in the target nucleotide sequence. The detection of the target variant nucleotide can be seen as detection of the target nucleotide sequence that contains it, i.e. as detection of a variant target nucleotide sequence. The target nucleic acid sequence is itself contained in a target nucleic acid molecule which is present in the sample. As discussed further below, the target nucleic acid sequence/molecule may be present as free nucleic acid in the sample, or it may present (i.e. in situ) in a cell or tissue in the sample. The target nucleotide sequence, or a copy or amplicon thereof, including a complementary copy, may be detected. In the present methods, the target variant is detected by detecting the presence or amount of the variant, and can include detecting simply if it is present or not, or any form of measurement indicative of the amount of variant present. Accordingly, detecting the target variant nucleotide/sequence includes determining, measuring, assessing or assaying the presence or absence or amount or location of the target variant in any way. The presence of a nucleotide sequence (i.e. the confirmation of its presence or amount) is indicative or identificatory of the presence of the target nucleic acid sequence.

Quantitative and qualitative determinations, measurements or assessments are included, including semi-quantitative. Such determinations, measurements or assessments may be relative, for example, when two or more different variant target nucleotide sequences in a sample are being detected, or absolute. Accordingly, in an embodiment, the method may be for quantifying or determining the amount of target variant nucleic acid sequence which is present. The term “quantifying” when used in the context of quantifying a target nucleic acid sequence(s) in a sample can refer to absolute or to relative quantification. Absolute quantification may be accomplished by inclusion of known concentration(s) of one or more control nucleic acid molecules and/or referencing the detected level of the target nucleic acid sequence with known control nucleic acid molecules or sequences (e.g. through generation of a standard curve). Alternatively, relative quantification can be accomplished by comparison of detected levels or amounts between two or more different target variant nucleic acid sequences, to provide a relative quantification of each of the two or more different nucleic acid sequences, i.e., relative to each other. Thus, as noted above, ratios of target variant nucleic acid sequences present in a sample may be determined. Thus, copy numbers of target variant nucleic acid sequences may be compared.

The target nucleic acid is typically DNA or RNA. However, it may also be composed of or may comprise other nucleic acids, natural or synthetic. Thus, it may, for example, be a chimeric construct comprising both RNA and DNA. The nucleic acid product may be made up of ribonucleotides and/or deoxyribonucleotides as well as synthetic nucleotides that are capable of participating in Watson-Crick type or analogous base pair interactions. Thus, the nucleic acid product may be or may comprise, e.g. bisulphite-converted DNA, LNA, PNA or any other derivative containing a non-nucleotide backbone.

In an embodiment, the target nucleic acid is DNA, natural or synthetic. The target nucleic acid may be coding or non-coding DNA, for example, genomic DNA or a sub-fraction thereof, or may be derived from genomic DNA, e.g. a copy or amplicon thereof, or it may be cDNA or a sub-fraction thereof, or an amplicon or copy thereof etc. The target nucleic acid may further be cell-free DNA in a sample, for example, foetal DNA in a maternal sample, or tumour DNA in a clinical sample (e.g. in blood or a blood-derived sample), or a copy of amplicon thereof. The target nucleic acid may be the nucleic acid directly as it occurs in the sample (e.g. the nucleic acid as present in situ in a sample), or it may derived from such nucleic acid, e.g. it may be a copy or amplicon thereof, including a complementary copy.

In another embodiment, the target nucleic acid is RNA. It may be an RNA molecule in a pool of RNA or other nucleic acid molecules, for example, genomic nucleic acids, whether human or from any source, from a transcriptome, or any other nucleic acid (e.g. organelle nucleic acids, i.e. mitochondrial or plastid nucleic acids), whether naturally occurring or synthetic. The target RNA may thus be or may be derived from coding (i.e. pre-mRNA or mRNA) or non-coding RNA sequences (such as tRNA, rRNA, snoRNA, miRNA, SIRNA, snRNA, exRNA, piRNA and long ncRNA). In an embodiment, the target nucleic acid molecule is a micro RNA (miRNA). In another embodiment, the target RNA molecule is 16S RNA, for example, wherein the 16S RNA is from and identificatory of a microorganism (e.g. a pathogenic microorganism) in a sample. Alternatively, the target RNA molecule may be genomic RNA, e.g. ssRNA or dsRNA of a virus having RNA as its genetic material. Notable such viruses include Ebola, HIV, SARS, SARS-COV2, influenza, hepatitis C, West Nile fever, polio and measles. Accordingly, the target RNA may be positive sense RNA, negative sense RNA, or double-stranded RNA from a viral genome, or positive-sense RNA from a retroviral RNA genome.

Where the target nucleic acid is RNA, the method may comprise a preliminary step of generating a cDNA copy of the target RNA.

The target nucleic acid sequence of the methods herein typically represents the assay target of the method, i.e. it is the target analyte of the method. It may be any target sequence, a variant of which it is desired to detect or identify. Thus, it may be any variant target sequence it is desired to detect, for example, in a nucleic acid present in a biological or clinical sample, or such like, e.g. a cell or tissue, or body fluid or liquid biopsy sample etc. It may thus be a naturally occurring sequence, or a derivative, or copy or amplicon thereof.

However, in another embodiment, the target nucleic acid sequence may be synthetic or artificial, for example, it may be a reporter sequence for an analyte of an assay. Reporter nucleic acids may be used or generated in the course of an assay for any analyte, for example, a protein or other biological molecule, or small molecule, in a sample. Thus, a reporter nucleic acid may be provided as a tag, or label, for a binding probe for an analyte, and may be detected in order to detect the analyte, for example, in an immunoassay, e.g. as in an immunoPCR or immunoRCA reaction. A reporter nucleic acid may be generated in the course of an assay, for example, by a ligation reaction in a proximity ligation assay (PLA), or an extension reaction in a proximity extension assay (PEA), or by a cleavage reaction, or such like. Such a reporter target nucleic acid may therefore be a synthetic or artificial sequence. It may be a linear or a circular or circularised or circularisable molecule.

As noted above, variants of target sequence may occur in various forms, such as, for example, mutations or polymorphisms etc. The variant nucleotide or variant nucleotide sequence may be a wild-type or mutant, or a polymorphism or allelic variant. Advantageously, the methods and uses herein may be used for the detection of mutations, including, for example, in clinical samples for the diagnosis, prognosis or study of disease etc.

Thus, the target nucleic acid sequence may be one of a number of different variants of the nucleic acid sequence which may occur in a target nucleic acid molecule. It is important to be able to distinguish different variants from one another, and thus for the signals generated for each variant to be able to be detected distinctly. For example, a sample may contain both mutant and wild-type sequences and it may be important to detect and quantify these separately, and this is also important to be able to determine allele frequencies correctly.

The target nucleic acid sequence may be present within a sample within which it naturally occurs, or it may be obtained from a sample, or present in a sample which is prepared. For example, nucleic acid, e.g. DNA may be extracted from a sample, and the extracted DNA may be subjected to amplification, to provide a sample for use in the methods herein. The sample may accordingly be any sample which contains any amount of nucleic acid, from any source or of any origin, in which it is desired to detect a variant target nucleic acid sequence. A sample may thus be any clinical or non-clinical sample, and may be any biological, clinical or environmental sample in which the target nucleic acid sequence may occur, or any sample which is prepared to contain such a sequence or a copy or amplicon thereof.

The sample may be any sample which contains a target nucleic acid molecule, and includes both natural and synthetic samples, that is, materials which occur naturally or preparations which have been made. Naturally occurring samples may be treated or processed before being subjected to the methods herein. All biological and clinical samples are included, e.g. any cell or tissue sample of an organism, or any body fluid or preparation derived therefrom, as well as samples such as cell cultures, cell preparations, cell lysates etc. Environmental samples, e.g. soil and water samples or food samples are also included. The samples may be freshly prepared or they may be prior-treated in any convenient way, e.g. for storage.

Representative samples thus include any material which may contain a target nucleic acid molecule, including, for example, foods and allied products, clinical and environmental samples. The sample may contain any viral or cellular material, including all prokaryotic or eukaryotic cells, viruses, bacteriophages, mycoplasmas, protoplasts and organelles. Such biological material may thus comprise all types of mammalian and non-mammalian animal cells, plant cells, algae including blue green algae, fungi, bacteria, protozoa etc., or a virus. The cells may be, for example, human cells, avian cells, reptile cells etc., without limitation.

Representative samples thus include whole blood and blood-derived products such as plasma, serum and buffy coat, blood cells, urine, faeces, cerebrospinal fluid or any other body fluids (e.g. respiratory secretions, saliva, milk, etc.), tissues, biopsies, cell cultures, cell suspensions, conditioned media or other samples of cell culture constituents, etc. The sample may be pre-treated in any convenient or desired way to prepare for use in the method, for example, by cell lysis or purification, isolation of the nucleic acid, etc.

In one embodiment, the sample comprises microbial cells or viruses which have been isolated from a clinical sample or from a culture of a clinical sample. In such a sample, the target nucleic acid molecule may be a nucleotide sequence present in a microbial cell, e.g. a nucleotide sequence which is characteristic for, or discriminatory or identificatory of a microbial cell or virus, at any level, e.g. at type, group, class, genus, species or strain level.

In another embodiment, the sample may contain cell-free DNA. The sample may be a sample such as plasma or serum which directly contains cell-free DNA, or the cell-free DNA may be isolated.

Since the target nucleic acid molecule need not itself be the target analyte of the assay, but can be a reporter molecule used or generated in the course of an assay for any desired analyte, the sample need not be a sample which naturally contains nucleic acid, or a source of nucleic acid (e.g. a cell or virus, or biological or clinical material etc.). As indicated above, the sample may be a synthetic or artificial sample. It may accordingly be a sample which has been subjected to a detection assay for an analyte in which a target nucleic has been generated, or to which a target nucleic acid molecule has been added. It may be a reaction mixture, or a reaction product, for example, the product resulting from an immunoassay to detect a target analyte, e.g. an immunoPCR, immunoRCA, or proximity assay (e.g. proximity ligation assay (PLA) or proximity extension assay (PEA).

In the methods herein, the target nucleotide sequence is contacted with a variant-specific probe. The term “contacting” is used broadly herein to include bringing the reagents in question into contact. Thus, one may be added to the other and vice versa, or they may each be introduced to each other etc. The contact may be within the initial sample in which the target nucleic acid occurs or is provided, or the target nucleic acid may be removed from the sample, and provided in another sample etc.

The variant-specific probe is designed to hybridise specifically to the target nucleotide sequence if the target variant nucleotide is present in that sequence. In other words, the variant-specific probe is capable of discriminating the target variant sequence from a non-target variant sequence, that is, from a different variant of that sequence. More particularly, the variant-specific probe comprises a nucleotide at its 3′ end which is capable of discriminating the target variant nucleotide from a non-target nucleotide.

The variant-specific probe is in effect a hybridisation probe. Such probes are widely known and used in the art, and described in the literature. They may take various forms, and may be detected in various different ways, e.g. by labels directly incorporated or provided in or on the probe, by detection sequences (e.g. barcode or tag sequences) in the probe, or by comprising binding sites for further detection reagents, such as e.g. primers, including amplification primers, or detection oligonucleotides which may themselves be labelled etc. Alternatively, the hybridisation probes may take part in further reactions (after the variant-specific hybridisation) which allow a signal for detection to be generated, for example, they make take part in ligation and/or extension (i.e. nucleic acid polymerisation) and/or cleavage reactions, wherein the probe, or a part thereof, is itself ligated or extended or cleaved, or in which it templates or enables a ligation and/or extension and/or cleavage reaction involving another oligonucleotide component or reagent. Indeed, the further detection reaction(s) may involve further hybridisation reactions, which ultimately build up a detectable hybridisation structure (e.g. branched DNA amplification reactions, based on principles such as underlie the RNAScope detection method, described further below). Such probes are also well-known and described in the art, and any such probe may be used. Indeed, the literature describes a vast array of different types of assays for detecting variant sequences using variant-specific probes with variant-specific nucleotides at their 3′ ends and any such probe or assay format may be used in the methods and uses herein.

Thus, as will be described in more detail below, the method may comprise or be based on a detection method involving rolling circle amplification (RCA) of a probe which either comprises a circular nucleic acid molecule which may be subject to RCA, or which is itself a circularisable oligonucleotide probe (e.g. a padlock probe, as described further below), or comprises or generates a circularisable component (e.g. by cleavage) which may in the course of the assay be circularised (e.g. by ligation) to create a circular template for RCA, or which comprises two or more parts which may be ligated together (e.g. an oligonucleotide ligation assay, OLA), or a hybridisation assay which involves ligation of hybridisation probes (e.g. into a backbone), or which involves an extension reaction, which includes, for example, molecular inversion probes, also known as gap-fill padlock probes, in which the gap is filled by extension of the hybridised 3′ end of the padlock probe, and the extended probe is then ligated to circularise it.

By way of representative example, the method may comprise or involve SuperRCA assay; FISH assay; OncoBEAM assay; competitive allele-specific Taqman PCR (Cast-PCR); Single-Nucleotide Primer Extension Assay (SNuPE); gap-fill padlock probe assay; RNAscope assay; or Basecope assay.

Accordingly, more generally, in an embodiment, the variant-specific probe is a hybridisation probe, and the hybridisation of the probe is detected. In another embodiment, the variant-specific probe is a ligation and/or extension probe, wherein a ligation and/or extension product of the probe, or an amplicon thereof, is detected.

Hence, in an embodiment, the method comprises a step of extension and/or ligation following hybridisation of the probe to the target nucleic acid sequence, optionally followed by a step of amplifying the ligation and/or extension product, and wherein the ligation, or extension, or ligation and extension, product of the probe, or an amplicon thereof, is detected.

The probe may comprise one or more parts, e.g. two or more parts. For example, it may comprise two or more separate oligonucleotides which, in use of the probe, are ligated together. Further, it may comprise two or more parts which are hybridised together, whether prior to, or in use. The probe may be subject to an intramolecular ligation (e.g. in the case of a padlock probe comprising a single circularisable oligonucleotide) or an intermolecular ligation (e.g. in the case of a 2-part padlock probe, for example, a gap-fill padlock probe wherein the gap is filled by a gap oligonucleotide, or a hybridisation assay wherein one or more hybridisation probes are ligated into a backbone).

The probe may comprise one or more binding sites for the target nucleotide sequence. Further, in the case of a probe with more than one part, one or more of said parts, including each part, may comprise one or more binding sites for the target nucleotide sequence.

As mentioned above, the probe may be detected in various ways, according to principles and methods well known in the art. Commonly, the detection of the probe may involve an amplification step, to increase the signal which is detected. This is particularly the case, where a nucleic acid product is generated from the operation of the probe, for example, a ligation and/or extension product as discussed above. In other words, the method may comprise a signal amplification step. Any amplification reaction may be used, including PCR and such like. RCA is particularly useful in this regard. RCA is an isothermal amplification technique which utilises a strand displacement polymerase enzyme and requires a circular amplification template. Amplification of the circular template provides a concatenated RCA product, comprising multiple copies of a sequence complementary to that of the amplification template. Such a concatemer typically forms a ball or “blob”, which may readily be visualised and detected, and thus RCA-based assays have been widely adopted for the detection of nucleic acids, where RCA is used to generate a detectable product.

Particularly advantageous are RCA-based assays which rely on secondary amplification of the initial RCA product (“RCP”), to increase the amount of product which is detected, and thereby to provide amplification of the signal in the assay. These include, for example, hyberbranched RCA which generates many unclustered subsequent RCA products through the strand displacement activity. More particularly, a “SuperRCA” (sRCA) reaction may be used, which comprises 2 or more rounds of RCA amplification, wherein the product of the second RCA reaction, the second RCP, is linked to that of the first, namely to the first RCP. WO 2015/071445 describes the basic principles of a sRCA reaction and its use in detection assays in general, and its disclosure is incorporated herein by reference. In such a method, a padlock probe is used to bind directly to the initial RCA product (RCP). Thus, a padlock probe may be hybridised to each monomer repeat of the first RCP (i.e. to each complementary copy of the first RCA template used to generate the first RCP), or at least to a high proportion thereof. The hybridised padlock probes may then be circularised by intramolecular ligation and serve as second RCA templates to generate a second RCP which is linked to the first.

Thus, in the methods herein, the variant-specific probe may be used to provide the template circle for a RCA reaction, or for the first or second RCA reaction of a sRCA reaction. Thus, in one preferred embodiment, the variant-specific probe is a circularisable probe, or in other words, a padlock probe. Padlock probes are well known in the art. Padlock probes may take many forms, and may be provided in 1-part form, or multi-part (e.g. 2-part) form. They include gap-fill padlock probes (also known as molecular inversion probes (MIPs)).

In the case where the variant-specific probe is a padlock probe, the method may comprise contacting the target nucleotide sequence (or more particularly a sample comprising the target nucleotide sequence) with the variant-specific padlock probe and allowing it hybridise to the target nucleic acid sequence. Hybridised padlock probes may then be circularised by ligation and detected. The detection may comprise amplifying the circularised padlock probes by RCA to form a RCA product (RCP) which is detected.

The variant-specific padlock probe may be used in the first or second RCA reaction of a sRCA reaction, for example, in which both RCA reactions use padlock probes or in which an amplicon of the target nucleic acid molecule comprising the target nucleic acid sequence is circularised. Hence, the target nucleotide sequence may be present in an amplicon of the target nucleic acid molecule. Thus, for example, in the method outlined above, the target nucleotide sequence may be present in a RCP generated by RCA of a padlock probe which is specific for the target nucleotide sequence in a sample, or which is capable of hybridising to, or to sequences in the target molecule which flank the target sequence. Alternatively, the target nucleic acid sequence may be present in a POR amplicon of the target nucleic acid molecule. Said PCR amplicon may be circularised, and subjected to sRCA reaction, as described further below.

The RCA template may alternatively be a pre-formed circle, which forms part of the variant-specific probe (e.g. is hybridised to a variant-specific probe, or to a nucleic acid part or domain thereof), or is used together with a variant-specific probe. Analogously, it may be a circularisable oligonucleotide which is ligated to form a circle during the course of the assay reactions.

In more detail, a padlock probe may, as noted above, alternatively be defined as a circularisable probe. The use of padlock or circularisable probes is well known in the art, including in the context of RCA reactions. A circularisable probe comprises one or more linear oligonucleotides which may be ligated together to form a circle. Thus, the principles of padlock probing are well understood and the design and use of padlock probes is known and described in the art. A padlock probe is typically a linear circularisable oligonucleotide which hybridizes to its target nucleic acid sequence or molecule in a manner which brings 5′ and 3′ ligatable ends of the probe into juxtaposition for ligation together, either directly or indirectly, with a gap in between. By ligating the hybridized 5′ and 3′ ends of the probe, the probe is circularized. It is understood that for circularization (ligation) to occur, the ligatable 5′ end of the padlock probe has a free 5′ phosphate group.

To allow the juxtaposition of the ends of the padlock probe for ligation, the padlock probe is designed to have the target-binding sites at or near its 5′ and 3′ ends. That is, the regions of complementarity which allow binding of the padlock probe to its target lie at or near the ends of the padlock probe.

To allow ligation, the 3′ and 5′ ends which are to be ligated (the “ligatable” 3′ and 5′ ends) are hybridized to the target sequence, which acts as the ligation template. The ligatable ends of a padlock probe may be brought into juxtaposition for ligation in various ways, depending on the probe design. Where the target-binding sites are located at the ends of the padlock probe, the binding of the padlock probe may bring the ends into said juxtaposition. Where the complementary binding sites in the target molecule or sequence lie directly adjacent (or contiguous) to one another, the ends of the padlock probe will hybridise directly adjacent to each other (i.e. with no gap) and may be ligated to each other directly. Thus, in this case the ligatable ends of the probe are provided by the actual ends of the probe. However, in an alternative configuration the padlock probe is a gap-fill padlock probe, and hence the binding sites at the ends of the padlock probe do not hybridise to adjacent binding sites, but rather to non-adjacent (non-contiguous) binding sites in the target sequence. In such an arrangement, the 5′ ligatable end of the probe is provided by the actual 5′ end of the probe. However, the ligatable 3′ end of the probe is generated by extension of the hybridized 3′ end of the probe, using the target sequence as extension template to fill the gap between the hybridized ends of the probe. The extension reaction brings the extended 3′ end of the probe into juxtaposition for ligation. In this case, the ligatable 3′ end of the probe is thus the extended 3′ end of the probe.

Padlock probes may be provided in 2 or more parts that are ligated together. This may involve the provision of an additional ligation template, for example, in the case of a 2-part probe, where each part comprises only one target-binding region and the other end of each part hybridizes to a common ligation template. In another embodiment, a 2-part padlock may take the form of a “connector” oligonucleotide with two target-binding regions at or near the 5′ and 3′ ends respectively, which hybridise to the target with a gap in between them, and a gap oligonucleotide which hybridizes in the gap between the ends. The gap oligonucleotide may partially or fully fill the gap.

In the variant specific padlocks probes herein, the 3′ end modified nucleotide lies at the 3′ end of the padlock, or at a part thereof which hybridizes to the target sequence at the position of the target variant nucleotide.

A detection method based on sRCA using gap-fill padlock probes is described in our co-pending application WO 2022/117769, and is particularly suited to the detection of variant target nucleic acid sequences, and is incorporated by reference herein. In an embodiment, the methods herein are particularly suited for use in the context of such a method. In the method a target-specific first padlock probe is used to “capture” a target sequence by hybridising to the target nucleic acid molecule and generating a complementary copy of the target sequence by a gap-fill extension reaction. The extended 3′ end of the first padlock probe is ligated to the 5′ end of the padlock probe to circularise it, and thereby generates the first RCA template. The circularised first padlock probe containing the complementary copy is then amplified by RCA to generate a first RCA product containing multiple copies of the target sequence. The resulting first RCA product is then probed with a further, second, padlock probe, specific for the target sequence. The circularised second padlock probes are subjected to a further, second RCA reaction, which is used to generate second RCA products attached to the first, and the resulting sRCA product is detected to detect the target sequence. The second padlock probe may be a variant specific-probe as described and used herein.

In an alternative version of such a sRCA-based assay, rather than using a padlock probe to generate a copy of the target sequence, amplicons of the target sequence may be prepared, conveniently by a PCR reaction, although any amplification method may be used. The amplicons are then circularised by ligation, conveniently but not necessarily using a ligation template, to generate a first RCA template. This is amplified by RCA, and thereafter the detection method continues as above to generate a sRCA reaction product which is detected. Such a method is described in our co-pending application GB 2203182.7. In such a method, the variant-specific probe is the padlock probe which is used to hybridise to the first RCA product.

Accordingly, in a particular embodiment, the detection method herein for detecting two or more target variant nucleotides in a target nucleotide sequence may comprise:
(a) providing a reaction mixture comprising amplicons of the target nucleotide sequence(s);
(b) ligating the 5′ and 3′ ends of single-stranded amplicons from (a) to circularise them;
(c) performing first RCA reactions using the circularised amplicons as first RCA templates to generate first RCA products (RCPs) comprising multiple repeats of a complementary copy of the target nucleic acid sequence in the amplicons;
(d) contacting the first RCPs with two or more different variant-specific padlock probes specific for the two or more target variant nucleotides and allowing the probes to hybridise to the complementary copies in the multiple repeats;
(e) directly or indirectly ligating the ends of the hybridised padlock probes which have hybridised to their target sequence complements (i.e. to the nucleotide sequence complements which are the targets of, or which correspond to the respective padlock probes) to circularise the hybridised padlock probes;
(f) performing second RCA reactions using the circularised variant-specific padlock probes as second RCA templates to generate second RCPs containing multiple repeat complementary copies of the circularised padlock probes, wherein different second ROPs are generated for each target variant nucleotide to be detected in the reaction mixture;
(g) detecting the second ROPs in the detection reaction mixture, thereby detecting the target variant nucleotides.

In step (e), correctly hybridised padlock probes are ligated, that is, those that have correctly and specifically hybridised to the complements of the target sequences comprising the target variant nucleotide that they are designed to detect (i.e. the padlock probes which have hybridised to their respective target sequence complements).

It will thus be understood that in step (d), the variant-specific padlock probes will hybridise to their intended target (i.e. specifically), where that target sequence comprising the target variant nucleotide is present.

Whilst padlock probes may be designed with target-specific binding regions which are specific for a particular target sequence, it will be understood that non-specific hybridisation may occur, particularly in the case of variant target sequences which are similar in sequence (e.g. where the variants are single nucleotide variants, such as SNPs, and such like). In the present methods, the modified 3′ end nucleotide of the variant-specific padlock probes acts to reduce such non-specific hybridisation. However, the high specificity of padlock probes also arises from the failure of such non-specifically hybridised padlock probes to be ligated (and hence any such non-ligated padlock probes would not be amplified by the subsequent RCA step). Thus, in the second ligation step of the detection method above, the padlock probes which have hybridised to their target sequence complements are ligated (that is, the padlock probes which have correctly hybridised to the complement of the target sequence comprising the target variant nucleotide they are intended to detect, or in other words, their corresponding or cognate, or respective target variant nucleotide).

In an embodiment, the amplicons in (a) are PCR amplicons. This includes any variant of PCR, including asymmetric PCR, for example.

In an embodiment, the method may include the step of performing the amplification reaction, e.g. PCR, to generate the amplicons. The amplicons of individual target sequences may be prepared together in multiplex, in a single reaction mixture, or they may be separately prepared in parallel, and then pooled or mixed to provide the reaction mixture of (a).

In an advantageous embodiment, the detection method, including as set out and discussed above, may be performed in a single reaction vessel.

sRCA methods such as those described above require a first RCA step, and at least a second RCA step. The method may comprise further RCA steps, to generate a third, or further generation RCA product, using third, or fourth padlock probes, and so on, each targeting the target nucleic acid sequence. The final generation RCA product may be detected.

Where the variant-specific probe is a padlock probe, it can be advantageous for the non-ligated padlock probes to be removed, or rendered inert, before they are detected. Since a ligated padlock probe is circularised, this may readily be achieved by degradation of un-ligated padlocks which remain in linear form, conveniently by exonucleolysis using an enzyme with exonuclease activity.

Since a 3′ end modified nucleotide may render a probe resistant to exonucleolytic digestion, this may conveniently be achieved using an enzyme with 5′ exonuclease activity. In an embodiment, the enzyme is a 5′ exonuclease, and in particular, lambda exonuclease. However, any 5′ exonuclease enzyme may be used.

As an alternative to padlock probes and detection by RCA, the variant-specific probe may be a hybridisation probe detected by a RNAScope™-type branched DNA amplification system based on hybridisation. RNAScope™ exemplifies a signal amplification system based on the build up of probes, or hybridisation oligonucleotides, which hybridise to one another, to provide multiple binding sites for labelled detection probes. RNAscope™ technology is described in WO2011/094669 for example. Whilst RNAscope™ was developed for in situ hybridisation for detection of RNA, it exemplifies the principle of using sandwich-type, or intermediate, hybridisation probes each providing multiple binding sites for labelled detection probes, to generate a detectable nucleic acid product comprising multiple labels.

The method may be carried out in heterogenous or homogenous formats. That is, it may be performed on a solid phase (or support), or in solution or suspension (i.e. without a solid phase or support), or indeed both, since a solid phase may be introduced at a later stage.

As noted above, the method may be carried out in multiplex, where two or more target variant nucleotides are detected at the same time, or as part of the same reaction, i.e. in the same reaction mixture. This will particularly be the case where two or more possible different variants are detected to detect which of the possible variant nucleotides are present, including e.g. wild-type, or which of one or more possible mutations. Thus, in such a case, two or more different variants of a particular target sequence may be detected (in this sense, although occurring in different variants, the target sequence may be thought of as the same sequence, or of representing the same target sequence, e.g. the variant sequences may correspond to the same target gene). However, it is also possible for a multiplex reaction to include two or more different target sequences in the sense of occurring in different target genes (i.e. they are not variants of each other), in each of which one or more different target variant nucleotides are detected.

The term “hybridisation” or “hybridises” as used herein refers to the formation of a duplex between nucleotide sequences which are sufficiently complementary to form duplexes via Watson-Crick base pairing, or any analogous base-pair interactions. Two nucleotide sequences are “complementary” to one another when those molecules share base pair organization homology. Hence, a region of complementarity in a molecule or probe or sequence refers to a portion of that molecule or probe or sequence that is capable of forming a duplex. Hybridisation does not require 100% complementarity between the sequences, and hence regions of complementarity to one another do not require the sequences to be fully complementary, although this is not excluded. Thus, the regions of complementarity may contain one or more mismatches. Accordingly, “complementary”, as used herein, means “functionally complementary”, i.e. a level of complementarity sufficient to mediate a productive hybridisation, which encompasses degrees of complementarity less than 100%. The degree of mismatch tolerated can be controlled by suitable adjustment of the hybridisation conditions. Those skilled in the art of nucleic acid technology can determine duplex stability empirically considering a number of variables including, for example, the length and base pair composition of the respective molecules or probe oligonucleotides, ionic strength, and incidence of mismatched base pairs, following the guidance provided by the art. Thus, the design of appropriate probes, or ligation templates or primers etc. for any of the reaction steps describe herein, and binding regions thereof, and the conditions under which they hybridise to their respective targets is well within the routine skill of the person skilled in the art.

A region of complementarity, such as, for example, to a target sequence in the binding region of a padlock probe, or between a detection sequence and a detection probe, or a RCA primer to the circularised padlock probe etc., may be at least 6 nucleotides long, to ensure specificity of binding, or more particularly at least 7, 8, 9 or 10 nucleotides long. The upper limit of length of the region is not critical, but may for example be up to 50, 40, 35, 30, 25, 20 or 15 nucleotides. A complementary region may thus have a length in a range between any one of the lower length limits and upper length limits set out above. In the case of a padlock probe, the length of an individual target-binding region may be in the lower ranges, so that the total length of the two binding regions when hybridised to their target is within the upper ranges. For example, an individual target binding region may be 8-15, e.g. 10-12 nucleotides, so that the total hybridised length is 16-30 nucleotides long, e.g. 20-24. It may be desirable, within the constraints of conformation of the probes, and spacing of the domains, and desired or favoured hybridisations, to minimise the total length of a padlock probe to minimise the size of the circle which is subjected to RCA, and hence to minimise the lengths of the complementary regions where possible.

The variant-specific probes, or products generated therefrom may be detected using any convenient protocol or detection modality. This may depend on the target sequence to be detected, the purpose of the method, and/or the specific details of the procedures employed in the method, and the labels used.

For instance, a RCP may be detected directly, e.g. the concatemer may be cleaved to generate monomers which may be detected using gel electrophoresis, or more typically the RCP may be detected by hybridising labelled detection oligonucleotides (which may alternatively be referred to as detection probes) that hybridise to the RCP. In this regard, the padlock probe used to generate the RCP may comprise a detection sequence, a complementary copy of which is copied into the RCP, and which provides a binding site for a detection oligonucleotide. The detection oligonucleotide need not, however, be directly labelled. For example, the detection oligonucleotide may be an unlabelled probe which functions as a sandwich probe. The concept of sandwich probes is well known in the art and may be applied according to any convenient protocol. The sandwich probes can bind to the RCP but are not directly labelled themselves; instead, they comprise a sequence to which labelled secondary oligonucleotides can bind, thus forming a “sandwich” between the RCP and the labelled secondary oligonucleotide.

A RCP may also be detected using non-sequence-specific nucleic acid labelling methods, e.g. DNA binding stains or dyes, which are widely known in the literature, or by using labelled nucleotides for incorporation into the RCP. Alternatively, the RCP may be detected indirectly, e.g. the product may be amplified by PCR and the amplification products may be detected.

Labelled probes or products may be detected using any of the well-established methods for analysis of nucleic acid molecules known from the literature including mass spectrometry, CyTOF, microscopy, real-time PCR, fluorescent probes, microarray, colorimetric analysis such as ELISA, flow cytometry, mass spectrometry, or by turbidometric, magnetic, particle counting, electric, surface sensing, or weight-based detection techniques.

Depending on the level of multiplexing, combinatorial labelling methods may be used, according to techniques well known in the art. For example, the large number of repeated sequences in the sRCA products can enable distinction amongst large numbers of such products via ratio labelling with fluorescent or other spectrophotometrically detectable probes. Such ratio-labelled detection probes may be used during flow cytometry, or microscopic detection techniques, e.g. imaging, to detect large numbers of sequences, e.g. the combination of at least two fluorophores at different ratios can lead to generation of multiple populations of fluorescent labels. For example, it has been found that using combinations of two fluorophores at different ratios, 7 different populations can be created. This may be expanded using 3- or 4-colour combinations.

Although various detection modalities may be employed, conveniently where the hybridised probes are subjected to further steps to generate detectable products, such products may be detected by microscopy or flow cytometry. In both cases, directly or indirectly labelled detection oligonucleotides may be used, for example, with fluorescent labels which may readily be detected. In particular, in a microscopy-based method, the labelled products (e.g. RCPs) may be detected by imaging.

The use of such detection techniques advantageously allow the nucleic acid products to be digitally recorded. Indeed, since the degree of signal amplification afforded by detection processes such as RCA, particularly sRCA, allows the products to be visualised, they may be detected by a camera or any device including a camera, such as a mobile phone.

To detect products generated in a homogenous format, they may be captured or brought down to a solid support, or surface, to facilitate imaging, or microscopic detection more generally. Particularly in the case of sRCA products, a second RCP, being a second generation RCA product, is larger and heavier and hence readily amenable to bringing down to a surface by centrifugation. Thus, for example, tubes or plates may readily be spun to bring second RCPs down to the bottom of the tube or of a well for detection by microscopy, and particularly imaging.

In multiplex procedures for detecting multiple different target variant nucleotides, the products resulting from the probes may be detected and distinguished by in situ sequencing, including, for example, sequencing by synthesis, sequencing-by-hybridisation and sequencing by ligation, next generation sequencing and/or sequential barcode decoding techniques, including by sequencing-by-synthesis, -ligation or -hybridisation.

As noted above, also provided herein are kits for performing the methods. The kits may include the variant-specific probes as discussed above, optionally together with one or more reagents and/or instructions for use of the kit. Such reagents include dNTPs, and polymerase and ligase enzymes, as well as primers, e.g. RCA primers. Further, components may include buffers or other reaction components for one or more of the various reactions. Still further optional components may include means or reagents for detecting the probe or reaction product thereof. This may include, for example, detection oligonucleotides and any necessary secondary labelling reagents, including, for example, as discussed above. Further optional components may include a solid support and/or means for capture and/or immobilisation of a target nucleic acid molecule, or of a reaction component. Instructions may be, for example, in printed form, or on a computer-readable medium, or as a website address.

Advantages of the methods herein are discussed above. Such advantages are particularly beneficial in the context of detecting a target sequence or variant in complex samples, or where they are present in low abundance. In embodiments in which the variant-specific probe is subject to or part of signal amplification reactions, e.g. in a RCA, or particularly sRCA reaction, the method is highly sensitive. The method is thus particularly suited to detecting or identifying very rare sequence variants. The method can be used, for example, to find and detect tumour-derived mutant DNA sequence in patient samples, including notably cell-free DNA in plasma. The method may thus find utility in the diagnosis or monitoring of cancer, or e.g. to reveal recurrence of the cancer. The method may be used in the context of any cell-free DNA, and may also find application in prenatal testing including particularly NIPT. The technique is rapid, has minimal instrument requirements, and allows multiplex analysis of sequence variants for enhanced sensitivity.

The method will now be described in more detail with reference to the Figures and the following non-limiting examples.

### EXAMPLES

### Materials and Methods

Extraction of genomic DNA. DNA was extracted from BM cells or of whole blood using the QIAamp DNA Blood mini kit (Qiagen cat. 51104) and eluted in 50 μL elution buffer.

### PCR Based Library Prep: Tube A

High fidelity PCR pre-amplification. Sequences of interest in genomic DNA were amplified with SuperFi DNA polymerase (Thermo Scientific) in 25 μL PCR reactions containing 1× SuperFi buffer, 0.2 mM dNTP. 100 nM Fwd/Rev PCR primers, 330 ng gDNA and 0.0005 U/μL SuperFi DNA polymerase. The PCR program was as follows: 98° C. for 30 sec, 10 cycles of 98° C. for 15 sec, 62° C. for 120 sec, and a final elongation at 72° C. for 5 min.

### SuperRCA Assay: Tube B

### Primer and PCR Polymerase Clean Up:

1 μL per target from the PCR based library prep was mixed with 20 μL clean-up solution containing 1× superRCA buffer (Rarity Bioscience AB), 0.125 μl Exol (Thermo Fisher Inc.), 0.0006U/μL Thermoliable Proteinase K. The mixtures were incubated at 37° C. for 10 min, followed by 55° C. for 10 min.

Ligase-mediated circularization of one strand of PCR products. 20 μL ligation solution containing 1× superRCA buffer (Rarity Bioscience), 100 nM of ligation template, complementary to both ends of one strand of the amplification products, 0.5 mM NAD (Sigma) and 2 U Ampligase (Lucigen) were added into the clean-up solution containing amplified PCR products. The mixtures were incubated at 95° C. for 1 min, followed by 58° C. for 30 min.

Target sequence amplification by a first RCA. Circularized strands of PCR products containing target nucleotide positions were amplified by RCA. 5 μL of 1× superRCA buffer (Rarity Bioscience), 1.8 mM dNTP (Invitrogen), 2.5 U Phi29 polymerase (New England Biolabs) 0.5 ug/μL BSA were added to the circularized products. The reactions were incubated at 37° C. for 30 min, then 65° C. for 10 min.

Genotyping of RCA products via padlock probe ligation. Padlock probes for genotyping (i.e. variant-specific padlock probes) were hybridized to first-generation RCA products and ligated in a sequence-specific manner, by adding 5 μL ligation mix containing 1× superRCA buffer (Rarity Bioscience), 3 mM NAD (Sigma), 2.5 U Ampligase (Lucigen), and 60 nM genotyping padlock probe pairs to the reaction mixtures, incubating at 55° C. for 30 min.

Digestion of the non-reacted genotyping probes. 5 μL clean-up solution containing 1× superRCA buffer (Rarity Bioscience AB), 1.2 μM primer and 1U/μL Lambda exo was added into the reaction mixture and incubated at 37° C. for 15 min, then 75° C. for 20 min.

Secondary RCA templated by padlock probes bound to primary RCA products. 5 μL RCA mixture containing 1× superRCA buffer (Rarity Bioscience), 0.6 mM dNTPs and 6 U Phi29 DNA polymerase (New England Biolabs) was added to the mixture and the reactions were incubated at 37° C. for 30 min.

Digital recording of SuperRCA products by flow cytometry. The final reaction mixtures containing SuperRCA products were diluted into hybridization buffer containing 100 nM fluorophore-labeled oligonucleotide probes specific for the different SuperRCA products, in 1× superRCA buffer (Rarity Bioscience) to a final volume of 250 μL. The solutions were applied onto the CytoFlex flow cytometer (Beckman Coulter) and SuperRCA products were counted at ‘Medium’ speed (30 μL/minute) for 150 seconds per sample.

### Example 1

### Allelic Distinction Using Phosphorothioate Bond Modified Probes

The allelic distinction performance of non-phosphorothioate bond modified probes (V4) in comparison to phosphorothioate bond modified probes (V42) was investigated. V4 probes and V42 probes have identical sequences, with the exception that the last base of a V42 probe is a phosphorothioate bond linked nucleotide. In the first experiment, the probes were used to detect mutant or wildtype sequences in the same IDH1 p.R132C sample. The results are shown in FIG. 1. In the 59° C. degree condition, V4 probe pairs (each comprising a V4 non-phosphorothioate bond modified probe for detecting a wild-type and a V4 non-phosphorothioate bond modified probes for detecting a mutant) demonstrated poor allele distinction on the wild-type population (population in the bottom right gate of each FACS plot). In contrast, while under the same condition, V42 probe pairs (each comprising a V42 phosphorothioate bond modified probe for detecting a wild-type and a V42 phosphorothioate bond modified probe for detecting a mutant) showed better allele distinction both for the mutant population (population in the left-sided gate of each FACS plot) and the wild-type population (population in the bottom right gate of each FACS plot).

V4 (unmodified) probes were also tested in the ligation temperature of 61° C. Although the population distinction was improved, both the signal intensity and events numbers were lower than for V42 (modified probes), indicating that while higher temperature could improve the allele distinction performance of the probes, this was at the cost of ligation efficiency. Apart from the indicated condition difference in FIG. 1, all three samples had the same conditions.

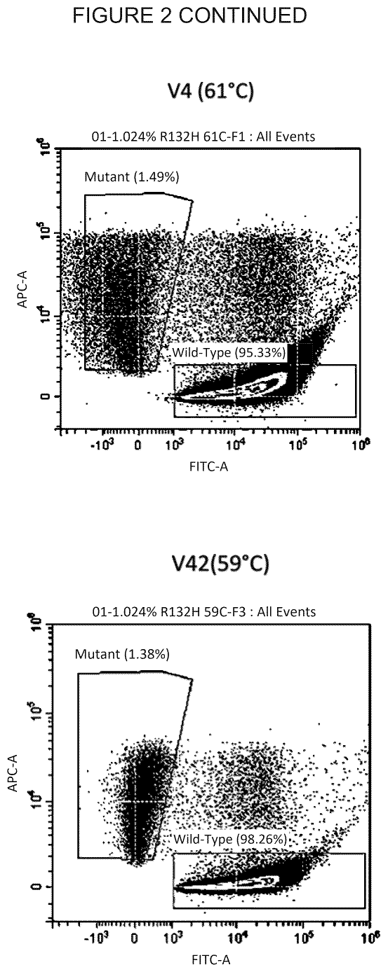

In the second experiment, the V4 and V42 probes were used to detect mutant or wildtype sequences in the same IDH1 p.R132H sample. The results are shown in FIG. 2. Similar to the results of the first experiment, in the 59° C. degree condition, V4 probe pairs (each comprising a V4 non-phosphorothioate bond modified probe for detecting a wild-type and a V4 non-phosphorothioate bond modified probes for detecting a mutant) demonstrated poor allele distinction on the wild-type population (population in the bottom right gate of each FACS plot). In contrast, while under the same condition, the V42 probe pairs (each comprising a V42 phosphorothioate bond modified probe for detecting a wild-type and a V42 phosphorothioate bond modified probe for detecting a mutant) showed better allele distinction both for the mutant population (population in the left-sided gate of each FACS plot) and the wild-type population (population in the bottom right gate of each FACS plot).

The V4 (unmodified) probes were also tested in the ligation temperature of 61° C. Although the population distinction was improved when compared to the temperature condition of 59° C., a significant number of wild-type probes were still falling out of the gate as compared to the results for the V42 (modified) probes. Apart from the indicated condition difference in FIG. 2, all three samples had the same conditions.

Results for FIG. 1:

### V4 (59° C.)

Tube Name: 01-1.024% R132C-G6 20220412 1.25/1.25 nM
Sample ID: 1.25 WT, 1.25 Mut
Volume (μl): 37.5

| | | | | Mean | Mean |
|---|---|---|---|---|---|
| Population | Events | % Total | % Parent | FITC-A | APC-A |
| All Events | 732920 | 100.00% | 100.00% | 18230.4 | 1717.3 |
| Wild-Type | 623178 | 85.03% | 85.03% | 12067.5 | 783.1 |
| Mutant | 11718 | 1.60% | 1.60% | −151.2 | 13442.8 |

### V4 (61° C.)

Tube Name: 01-2.048%-G5 20220419 2.5/2.5 nM
Sample ID: R132C
Volume (μL): 37.5

| | | | | Mean | Mean |
|---|---|---|---|---|---|
| Population | Events | % Total | % Parent | FITC-A | APC-A |
| All Events | 633917 | 100.00% | 100.00% | 15124.3 | 545.6 |
| Wild-Type | 618362 | 97.55% | 97.55% | 14192.1 | 268.3 |
| Mutant | 9651 | 1.52% | 1.52% | 10.4 | 10567.0 |

### V42 (59° C.)

Tube Name: 01-1.024%-G5 20220411 2.5/1.25 nM
Sample ID: R132C
Volume (μL): 37.5

| | | | | Mean | Mean |
|---|---|---|---|---|---|
| Population | Events | % Total | % Parent | FITC-A | APC-A |
| All Events | 842104 | 100.00% | 100.00% | 25348.3 | 700.6 |
| Wild-Type | 820913 | 97.48% | 97.48% | 23766.4 | 337.9 |
| Mutant | 12460 | 1.48% | 1.48% | −81.8 | 13577.8 |

Results for FIG. 2:

### V4 (59° C.)

Tube Name: 01-1.024% R132H 59C-F1 20220421 1.25/1.25 nM
Sample ID: V4 1.25_1.25
Volume (μL): 37.5

| | | | | Mean | Mean |
|---|---|---|---|---|---|
| Population | Events | % Total | % Parent | FITC-A | APC-A |
| All Events | 1020462 | 100.00% | 100.00% | 17833.2 | 3026.5 |
| Wild-Type | 624923 | 61.24% | 61.24% | 7826.9 | 941.7 |
| Mutant | 15720 | 1.54% | 1.54% | −418.0 | 22115.0 |

### V4 (61° C.)

Tube Name: 01-1.024% R132H 61C-F1 20220421 1.25/1.25 nM
Sample ID: V4 1.25_1.25
Volume (μL): 37.5

| | | | | Mean | Mean |
|---|---|---|---|---|---|
| Population | Events | % Total | % Parent | FITC-A | APC-A |
| All Events | 959002 | 100.00% | 100.00% | 22589.4 | 1259.5 |
| Wild-Type | 914233 | 95.33% | 95.33% | 20534.5 | 517.4 |
| Mutant | 14335 | 1.49% | 1.49% | −393.3 | 23837.9 |

### V42 (59° C.)

Tube Name: 01-1.024% R132H 59C-F3 20220421 1.25/1.25 nM
Sample ID: V42 1.25_1.25
Volume (μL): 37.5

| | | | | Mean | Mean |
|---|---|---|---|---|---|
| Population | Events | % Total | % Parent | FITC-A | APC-A |
| All Events | 847094 | 100.00% | 100.00% | 16093.9 | 468.0 |
| Wild-Type | 832395 | 98.26% | 98.26% | 15846.5 | 237.3 |
| Mutant | 11650 | 1.38% | 1.38% | 150.4 | 13066.7 |

1. A method of detecting a target variant nucleotide in a target nucleotide sequence in a sample, said method comprising contacting said target nucleotide sequence with a variant-specific padlock probe capable of hybridising to said target nucleotide sequence, and detecting said probe in order to detect said target variant nucleotide wherein the variant-specific padlock probe comprises a variant-specific nucleotide at a 3′ end thereof, and wherein said variant-specific 3′ nucleotide is a modified nucleotide.

2. The method of claim 1, wherein the modified nucleotide comprises a modification which alters its binding affinity to the nucleotide in the target nucleic acid sequence at the position of the variant nucleotide.

3. The method of claim 1 or 2, wherein the padlock probe comprises one or more additional modified nucleotides, in addition to the 3′ end modified nucleotide.

4. The method of claim 1 or 2, wherein the padlock probe does not comprise any further modified nucleotides beyond the 3′ end modified nucleotide.

5. The method of any one of claims 1 to 4, wherein the modified nucleotide comprises one or more of the following:
(a) a modification in the backbone;
(b) a modification in the sugar; and
(c) a modification in the base.

6. The method of claim 5, wherein:
(i) the modification in the backbone comprises phosphorothioate; phosphorothiolate; boranophosphate; phNA; or PNA;
(ii) the modification in the sugar comprises 2′F RNA; 2′OMe RNA; LNA; FANA; HNA; or 2′-O-methoxy-ethyl(2′ MOE);
(iii) the modification in the backbone and sugar comprises mirror DNA; ribuloNA; TNA; tPhoNA; or dXNA; or
(iv) the modification in the base comprises C7-modified deaza-adenine; C7-modified deaza-guanosine; C5-modified cytosine; C5-modified uridine; deoxyinosine; 5-hydroxybutynyl-2′-deoxyuridine (Super T®); 8-aza-7-deazaguanosine (Super G®); 2,6-diaminopurine (Z).

7. The method of any one of claims 1 to 6, wherein the modified nucleotide comprises:
(a) phosphorothioate or phosphorothiolate;
(b) LNA;
(c) 2′-O-methoxy-ethyl(2′MOE);
(d) 2,6-diaminopurine (Z);
(e) deoxyinosine;
(f) 5-methyl dC;
(g) 5-hydroxybutynyl-2′-deoxyuridine (Super T®); or
(h) 8-aza-7-deazaguanosine (Super G®).

8. The method of any one of claims 1 to 7, wherein the target variant nucleotide is a wild-type nucleotide, a mutant target nucleotide, an allelic variant, or a polymorphism.

9. The method of any one of claims 1 to 8, wherein the target nucleic acid sequence is genomic DNA.

10. The method of any one of claims 1 to 9, wherein the target nucleotide sequence is comprised in a cell-free DNA molecule.

11. The method of any one of claims 1 to 10, wherein the sample is, or is prepared from, a clinical sample.

12. The method of any one of claims 1 to 11, wherein the sample is a liquid biopsy sample.

13. The method of any one of claims 1 to 12, wherein the sample is a serum or plasma.

14. The method of any one of claims 1 to 13, wherein the sample is a cell or tissue sample and the target variant nucleotide is detected in situ.

15. The method of any one of claims 1 to 14, wherein the method is a multiplexed method for detecting two or more different target variant nucleotides at a target position in the target nucleotide sequence, and comprises contacting the sample with two or more different variant-specific padlock probes, one for each target variant nucleotide to be detected, and wherein a 3′ end nucleotide of at least one of said variant-specific padlock probes is modified.

16. The method of claim 15, wherein the 3′ end nucleotide of at least one of said variant-specific padlock probes is modified such that the 3′ end nucleotide of each of the different variant-specific padlock probes has a similar affinity to its target variant nucleotide in the target nucleic acid sequence.

17. The method of any one of claims 1 to 16, wherein:
(i) the hybridisation and ligation of the padlock probe or of an amplicon thereof is detected; or
(ii) the padlock probe is a gap-fill padlock probe, and a ligation or extension and ligation product of the padlock probe, or an amplicon thereof, is detected.

18. The method of any one of claims 1 to 17, wherein:
(i) the padlock probe comprises one or more parts; and/or
(ii) the padlock probe or one or more parts thereof comprises one or more binding sites for the target nucleotide sequence.

19. The method of any one of claims 1 to 18, wherein the padlock probe is a two-part padlock probe.

20. The method of any one of claims 1 to 19, wherein the method comprises a step of hybridisation of the padlock probe to the target nucleic acid sequence, optionally a step of extension of a hybridised 3′ end of the padlock probe, followed by ligation of the padlock probe, optionally further followed by a step of amplifying the ligation, or extension and ligation product, and wherein the ligation, or ligation and extension, product of the padlock probe, or an amplicon thereof, is detected.

21. The method of any one of claims 19 and 20, wherein padlock probes which have been circularised by ligation, optionally following a gap-fill extension step, are subjected to RCA, and the RCA products are detected.

22. The method of any one of claims 19 to 21, wherein padlock probes which have not been circularised by ligation are degraded using an enzyme with 5′ exonuclease activity.

23. The method of claim 22, wherein the enzyme with 5′ exonuclease activity is lambda exonuclease.

24. Use of a variant-specific padlock probe to detect a target variant nucleotide in a target nucleotide sequence, wherein the variant-specific padlock probe comprises at least one target-binding site that is complementary to the target nucleotide sequence and is capable of hybridising thereto, and wherein said target binding site lies at a 3′ end of the padlock probe and comprises a variant-specific nucleotide at the 3′ end which is a modified nucleotide.

25. A variant-specific padlock probe comprising at least one target-binding site that is complementary to a target nucleotide sequence and is capable of hybridising thereto, wherein said target binding site lies at a 3′ end of the probe and comprises a variant-specific nucleotide at the 3′ end which is capable of base-pairing with a target variant nucleotide in the target nucleotide sequence, and which is a modified nucleotide.

26. A kit for use in the method of any one of claims 1 to 23, said kit comprising a plurality of variant-specific padlock probes, each specific for a different variant at a target position in a target-nucleotide sequence, wherein at least one variant-specific padlock probe comprises a modified nucleotide at a 3′ end thereof.
